(19) United States
(12) Patent Application Publication
(10) Pub. No.: US 2024/0041478 A1
(43) Pub. Date: Feb. 8, 2024

(54) GUIDE DEVICE FOR BONE CORRECTION PROCEDURE AND BONE CORRECTION METHOD

(71) Applicants: Gustavo Araujo NUNES, Brasília-DF (BR); GM dos Reis Indústria e Comércio Ltda., Campinas-SP (BR)

(72) Inventors: Gustavo Araujo NUNES, Brasília-DF (BR); Geraldo Marins DOS REIS, JR., Campinas (BR)

(21) Appl. No.: 17/816,874

(22) Filed: Aug. 2, 2022

(51) Int. Cl. A61B 17/17

(52) U.S. Cl. CPC A61B 17/1775; A61B 17/1796; A61B 2017/00477

(57) ABSTRACT

The present invention refers to a guide device for bone correction procedure and a method for bone correction. The device includes a first spacing element, a second spacing element, at least one connecting rod, a first graded element, and a second graded element. Each one of the first spacing element and the second spacing element have at least one through hole. The at least one connecting rod is coupled to the at least one through hole of the first spacing element and to the at least one through hole of the second spacing element. The first graded element and the second graded element are each respectively coupled to the first spacing element and to the second spacing element. The first graded element and the second graded element each have at least one through hole associated to at least one grading.

### FIELD OF THE INVENTION

The present invention refers to a guide device able to assist a surgeon during a bone correction procedure in a patient. The present invention further refers to a bone correction method. Particularly, the device of the present invention is able to assist a surgeon during a minimally invasive procedure.

### BACKGROUND OF THE INVENTION

Hallux valgus, commonly known as bunion, is a physiopathology that is characterized by metatarsal bone deformity, specifically affecting the metatarsal phalangeal joint system I (MTP I).

Several etiological studies indicate that the hallux valgus develops, in most of the cases, due to extrinsic factors, such as the prolonged use of anatomically inadequate shoes, which results in the action of valgus forces that contribute to the progressive mid-lateral compression of the hallux and the toes.

However, intrinsic factors can, less frequently, be the cause of the development of hallux valgus, such as congenital valgus of the first metatarsal bone, differences in the length of the hallux relative to the toes and the evolution of neuromuscular and rheumatic diseases that affect the joint structure of the feet, favoring the development of hallux valgus.

In its initial development stage, the proximal phalanx of hallux deviates laterally from the head of the first metatarsal bone in the valgus direction to the second toe. In response to this deviation, the head of the first metatarsal bone also undergoes progressive displacement but in the varus direction of the second toe, while the tendon of the abductor hallucis muscle prevents the sesamoid bones from accompanying the displacement of the head of the first metatarsal bone.

In cases where the hallux presents a valgus angle greater than 35°, the abductor hallucis muscle enhances the pronation, or rotation of same relative to the first metatarsal bone, and with the varus displacement of the head of the first metatarsal bone by the lateralization of the proximal phalanx, there is developed a medial eminence noticeable as a bony bump, popularly known as bunion.

With the progression of this physiopathology, there arise other complications, such as visible subluxation of the sesamoids, as well as bone enlargement on the medial face of the head of the first metatarsal bone.

With the evolution of the condition of the hallux valgus, the advanced valgization of the first proximal phalanx to the hallux, and the plantar flexion of the head of the first metatarsal bone can result in sub position or over position of the hallux on the second toe. In this phase of the evolution of the condition, there occurs the complete impairment of the individual's biomechanics, since the plantar strength distribution is compromised, which in the long run is harmful to several joints throughout the individual's body.

At this point, apart from the bone deformity being visually perceivable, the affected individual can feel pain, redness, and warmth in the region of the metatarsophalangeal joint I (MTP I), as a severe inflammatory process begins, as well as callus formation due to the poor distribution of the plantar force, thickening of the skin in the region of the medial eminence and the emergence of bursas, apart from the progressive stiffening of the hallux joints as a whole.

As a solution to the hallux valgus condition, there exist several conservative treatments and minimally invasive surgical techniques that can vary according to the patient's expectation and the severity of the physiopathological condition.

As examples of minimally invasive surgical techniques, we can cite the Chevron osteotomy, the Scarf osteotomy, the metatarsophalangeal arthrodesis and the Lapidus arthrodesis.

Another very widespread technique is the percutaneous distal osteotomy of the first metatarsal bone, with metatarsal head rotation. In this technique, at a preoperative step, the surgeon assesses the bone deformity and the degree of rotation of the head of the first metatarsal bone by means of X-rays or tomography with load, so, when starting the intraoperative step, the surgeon will know how many degrees to turn the head, to provide the correct alignment of the bone.

This preoperative assessment of the rotation degree of the head of the first metatarsal bone, as well as the control of the rotation for the alignment of same during the intraoperative step is essential to avoid an extension of the metatarsophalangeal joint system I (MTP I) in the post-operative step, which can cause a relapse of the deformity in the long term.

In this sense, it is important to mention that, despite the use of image enhancers and fluoroscopes during the intraoperative step, said equipment does not generate accurate pictures for accompanying the rotation of the head of the first metatarsal bone.

Thus, after performing the distal osteotomy of the first metatarsal bone, the surgeons must perform manual surgical procedures to rotate the head of the first metatarsal bone, which generates extreme inaccuracy of the necessary rotation, resulting in angulation that is higher or lower than planned, apart from increasing the surgery time and the postoperative risks.

In this sense there exist several solutions of the state of the art in the orthopedic field which intend to assist the orthopedic surgeon during a hallux valgus correction surgery, but none of them is intended to guide the surgeon during the rotation and correct alignment of the head of the first metatarsal bone.

For example, the prior art available at https://www.youtube.com/watch?v=qVBHdHMOiGQ and published on Sep. 22, 2016, illustrates a Lapidus arthrodesis procedure wherein two metallic guide wires are respectively inserted in the proximal region of the first metatarsal bone and in the medial cuneiform bone to receive a device having the function of providing support to said bones while the arthrodesis of the first metatarsal cuneiform joint arthrodesis is performed, subsequently using bone screws and an Ortholoc™ 3di Crosscheck™ bone plate for the osteosynthesis.

Further, the prior art available at https://www.youtube.com/watch?v=sF83q6tWV2c and published on Jun. 19, 2019, illustrates the Lapiplasty® 3D Bunion Correction™ surgical procedure. In summary, the referred surgical procedure involves the use of a series of guide devices to guide the surgeon during the first metatarsal-cuneiform joint arthrodesis.

As can be observed, none of the prior art applies to a distal first metatarsal bone percutaneous osteotomy with rotation of the metatarsal head, nor does it provide means to align the head of the first metatarsal bone.

In view of the above, it is clear that the state of the art lacks technological improvements in the orthopedic field to assist a surgeon in a hallux valgus correction surgery, specifically for the rotation and correct alignment of the head of the first metatarsal bone.

### OBJECTIVES AND DESCRIPTION OF THE INVENTION

Thus, a general purpose of the present invention is to provide a guide device for bone correction procedure able to eliminate or at least reduce the limitations known from the current state of the art.

A particular purpose of the present invention is to provide a device capable of assisting the surgeon during the correction of a bone to be corrected relative to a reference bone in a minimally invasive surgery, and that results in reduction of the surgery time and avoids a relapse of the deformity in the postoperative period.

Additionally, it is a general purpose of the present invention to provide a bone correction method able to eliminate or at least reduce the limitations known from the current state of the art.

A particular purpose of the present invention is to provide a bone correction method which comprises means to assist the surgeon during the correction of a bone to be corrected relative to a reference bone in a minimally invasive surgery, and which results in reduction of the surgery time and avoids a relapse of the deformity in the postoperative period.

One or more purposes of the above mentioned invention, among others is(are) reached by means of a guide device for bone correction procedure, comprising:
a first spacing element and a second spacing element, each one of the first spacing element and the second spacing element having at least one through hole;
at least one connecting rod coupled to the at least one through hole of the first spacing element and to the at least one through hole of the second spacing element; and
a first graded element and a second graded element, each one being respectively coupled to the first spacing element and the second spacing element, each one of the first graded element and the second graded element having at least one through hole associated to at least one grading.

One or more purposes of the present invention mentioned above, among others, is(are) also reached by means of a bone correction method, comprising the steps of:
assessing the rotation degree of a bone to be corrected;
positioning a first graded element of a first spacing element of a guide device for bone correction on a reference bone;
positioning a second graded element of a second spacing element of the guide device for bone correction on the bone to be corrected;
inserting a first metallic guide wire in a first through hole of the first graded element having a reference angulation until the first metallic guide wire anchors on the reference bone;
inserting a second metallic guide wire in a second through hole of the second graded element which has an angulation compatible with the assessed rotation degree of the bone to be corrected until the second metallic guide wire anchors on the bone to be corrected;
removing the guide device for bone correction;
performing an osteotomy procedure in the bone to be corrected;
rotating the second metallic guide wire until it is visually aligned to the first metallic guide wire; and
reinserting the first metallic guide wire and the second metallic guide wire in the guide device for bone correction, wherein the first metallic guide wire is reinserted in the first through hole of the first graded element of the guide device for bone correction having the first reference angulation, and wherein the second metallic guide wire is inserted in a second through hole of the second graded element of the guide device for bone correction in a second reference angulation discounting the corrected rotation, and therefore, correcting the rotation of the bone to be corrected.

### BRIEF DESCRIPTION OF THE DRAWINGS

The objectives, technical effects and advantages of the present invention will be clear to those skilled in the art from the following detailed description which refers to the attached figures which illustrate exemplary embodiments, but not limiting, of the claimed objects:

FIG. 1 illustrates a perspective view of the guide device 300 for bone correction procedure, according to one embodiment of the present invention;

FIG. 2A illustrates an upper view representative of the first graded element 11a and of the second graded element 11b, according to one embodiment of the present invention;

FIG. 2B illustrates a longitudinal sectional view representative of the first graded element 11a and of the second graded element 11b, according to one embodiment of the present invention;

FIG. 3A illustrates a perspective view of part of the guide device 300 without the graded elements, according to one embodiment of the present invention;

FIGS. 3B, 3C and 3D illustrate, respectively, a back, front and side right view of part of the guide device 300 without the graded elements, according to one embodiment of the present invention;

FIGS. 4A and 4B illustrate, respectively, a side right view and a side left view of the first spacing element 10a and of the second spacing element 10b, with dashed lines indicating the internal configurations thereof, according to one embodiment of the present invention;

FIG. 5 illustrates a longitudinal sectional view representative of the attachment elements 50a, 50b, according to one embodiment of the present invention;

FIG. 6 illustrates a front view of the connecting rod 14b, according to one embodiment of the present invention;

FIGS. 7A and 7B illustrate, respectively, a right side view of the handle 15, with dashed lines indicating its internal configuration, and a longitudinal sectional view of same, according to the present invention;

FIG. 8 illustrates a perspective view of the at least one support rod 14a, 14c, according to the present invention;

FIG. 9 illustrates, respectively, a back view and front view of the first metallic guide wire 18a and the second metallic guide wire 18b, according to the present invention; and

FIG. 10 illustrates the steps of method 400 of bone correction, according to one embodiment of the present invention.

### DESCRIPTION OF EMBODIMENTS OF THE INVENTION

Initially, it must be emphasized that the device 300 and the method 400 of the present invention will be described as follows in accordance with particular embodiments, but not limitative, since the embodiments may be executed in different manners and variations and according to the application desired by the person skilled in the art.

In one embodiment, the present invention discloses a guide device 300 for bone correction that is able to guide a surgeon during the rotation and correct alignment of the head of the first metatarsal bone in a minimally invasive surgery.

In another embodiment, the present invention discloses a method 400 for bone correction comprising means to guide the surgeon during the rotation and correct alignment of the head of the first metatarsal bone in a minimally invasive surgery.

In particular, the minimally invasive surgery refers, preferably, to the correction of hallux valgus. However, a person skilled in the art will immediately notice that the device 300 and the method 400 can be used in any other bone correction procedure, in any bone to be corrected.

In the context of the present invention, the expression “bone correction” refers, preferably, to the correction of the head of the first metatarsal bone plantar flexed, but it must be also understood as any bone able to be corrected by a surgeon, guided by the guide device 300.

Additionally, the expression “procedure” refers, preferably, to a distal percutaneous osteotomy of the first metatarsal bone with rotation of the metatarsal head but must also be understood as any surgical procedure to which the guide device 300 can be applied.

Further, as regards the step of method 400 wherein a second metallic guide wire 18b is inserted in a second through hole of the graded element 10b, which has an angulation compatible with the assessed rotation degree of the bone to be corrected, the expression “compatible” refers, preferably, to the angulation of said through hole that receives the second metallic guide wire 18b, being equal or as close as possible to the assessed degree of rotation of the bone to be corrected.

For example, in one embodiment wherein the bone to be corrected was assessed in a 35° rotation, but the maximal angulation available for said through hole which receives the second metallic wire guide 18b is of 30°, it would still be possible to carry out the method 400 choosing the closest angulation to the assessed degree of rotation.

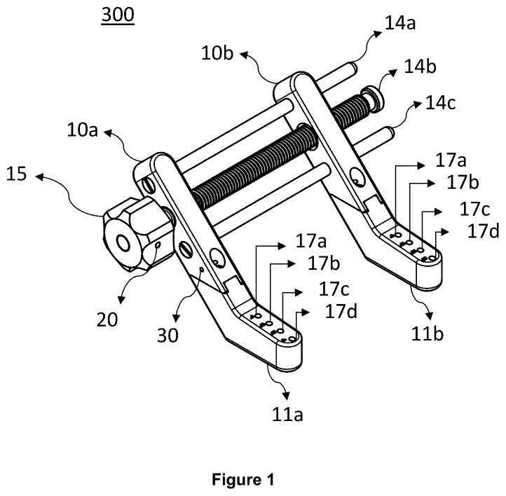

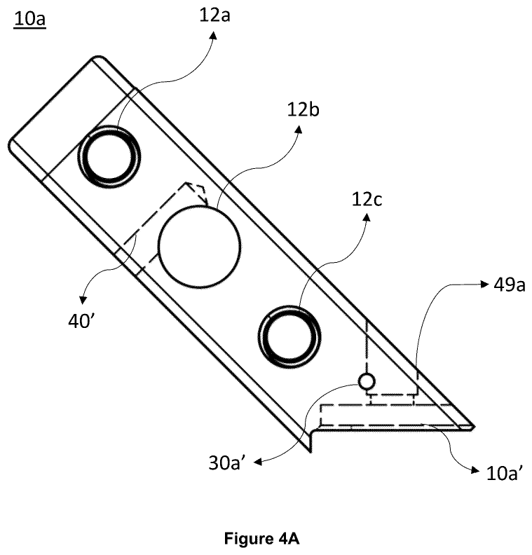

In one embodiment, and as can be seen in FIGS. 1, 4a and 4b, the guide device 300 for bone correction procedure comprises a first spacing element 10a and a second spacing element 10b, wherein each one of the first and second spacing element 10a, 10b has an oblong profile and at least one through hole 12a, 13a, 12b, 13b, 12c, 13c. In this embodiment, the first spacing element 10a has through holes 12a, 12b, 12c, while the second spacing element 10b also has three through holes 13a, 13b, 13c. Further, in this embodiment, both through holes 12a, 12b, 12c and 13a, 13b, 13c are located, respectively, on the side faces of the first spacing element 10a and of the second spacing element 10b. However, in an alternative embodiment, it will be apparent to a person skilled in the art that said through holes 12a, 12b, 12c and 13a, 13b, 13c can be arranged on the front faces of each one of the first spacing element 10a and the second spacing element 10b.

Further, referring to FIGS. 1, 4a and 4b, when the guide device 300 is in its assembled configuration, at least one correcting rod 14b is coupled to one of the through holes 12a, 12b, 12c, of the first spacing element 10a and to one of the through holes 13a, 13b, 13c, of the second spacing element 10b, so that the geometric centers of said through holes 12a, 12b, 12c and 13a, 13b, 13c are substantially aligned with each other.

In a preferred embodiment, the connecting rod 14b is coupled between the through hole 12b of the first spacing element 10a and between through hole 13b of the second spacing element 10b.

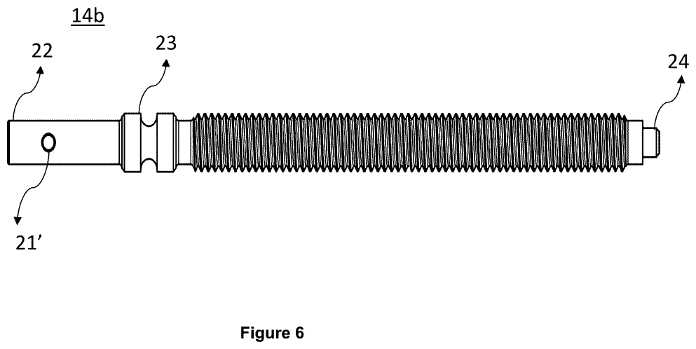

In this embodiment, and in accordance with FIGS. 4a and 4b, the through hole 12b of the first spacing element 10a, has a smooth inner profile, while the through hole 13b of the second spacing element 10b, has a threaded inner profile. Accordingly, and in accordance with FIG. 6, the connecting rod 14b has a first end 22 a second end 24, a threaded portion along part of the length thereof, as well as a smooth portion having a grooved profile 23.

In this manner, when the guide device 300 is in its assembled configuration, the first spacing element 10a is inserted in the first end 22 of the connecting rod 14b by means of the through hole 12b until the smooth inner profile of said through hole 12b is in contact with the portion with grooved profile 23. In a similar manner, the second spacing element 10b is inserted in the second end 24 of the connecting rod 14b by means of through hole 13b so that the threaded inner profile of said through hole 13b is in contact with the threaded portion of the connecting rod 14b.

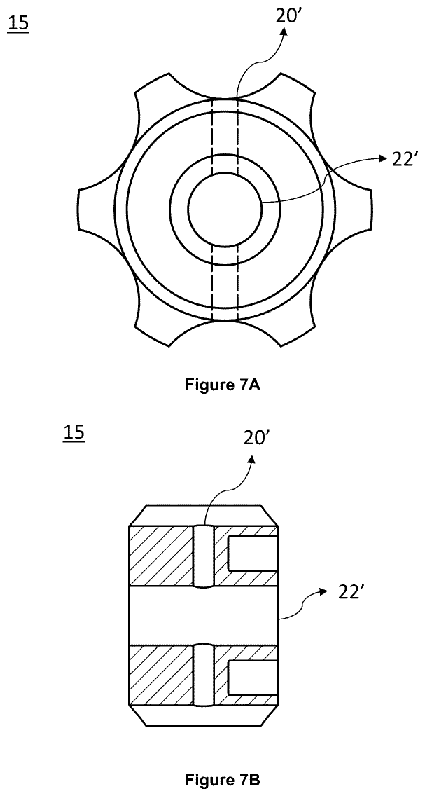

Still in connection with the connecting rod 14b, as can be seen from FIGS. 1, 6, 7A, and 7B, the first end 22 of the connecting rod 14b is adapted to receive a handle 15. In one embodiment, the handle 15 has a first through hole 22′ and a second through hole 20′ substantially perpendicular to the first through hole 22′, while the first end 22 of the connecting rod 14b has a through hole 21′ in the transverse direction of the first end 22.

In this manner, when the guide device 300 is in its assembled configuration, the first through hole 22′ of the handle 15 is inserted in the first end 22 of the connecting rod 14b until the second through hole 20′ of the handle 15 is aligned with the through hole 21′ in the transverse direction of the first end 22.

Once the referred alignment is reached, the handle is fixed to the first end 22 of the connecting rod 14b by means of the insertion of a first lock element 20 through the second through hole 20′ of the handle and of through hole 21′ in the transverse direction of the first end 22.

In this embodiment, the first spacing element 10a is arranged at a proximal end relative to the handle 15, while the second spacing element 10b is arranged at a distal end relative to the handle 15.

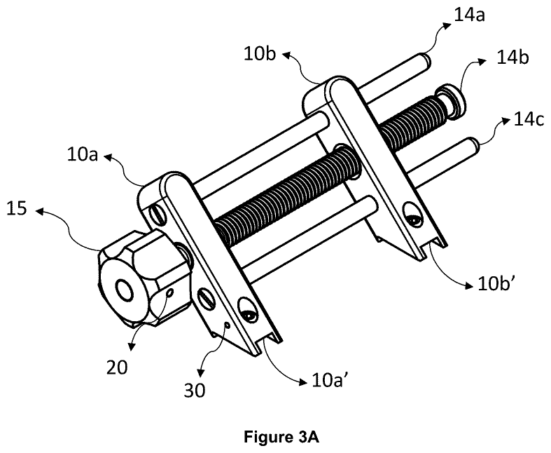

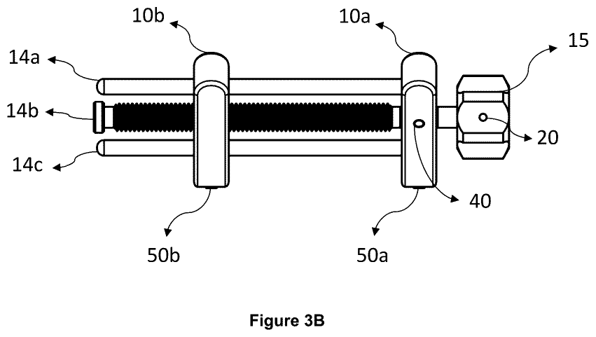

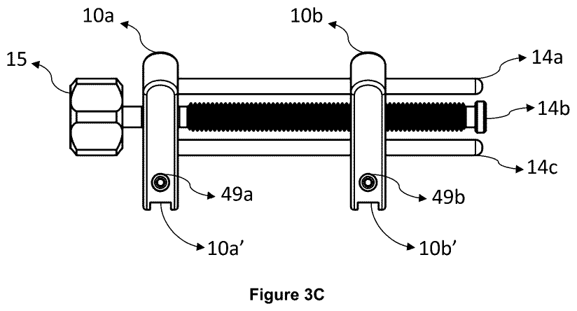

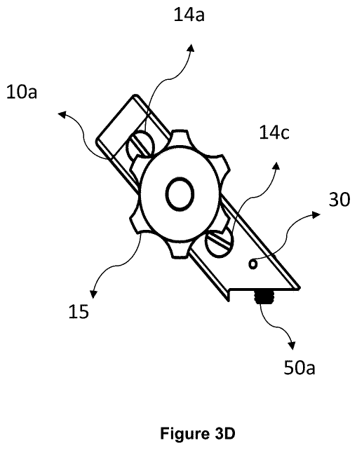

In one embodiment, and as can be visualized by means of FIGS. 3B and 4A, the first spacing element 10a has a non-through hole 40′ on its back face. The non-through hole 40′, is substantially perpendicular to the through hole 12b so that when inserting the second lock element 40 in the non-through hole 40′, part of the second lock element 40 enters the cavity formed by the through hole 12b. Consequently, part of the second lock element 40 rests on the portion with grooved profile 23 of the connecting rod 14b.

In this manner, the second lock element 40 rests on the portion with grooved profile 23 of the connecting rod 14b advantageously allows the connecting rod to turn on torque when a force is applied on the handle 15.

Thus, a force applied to handle 15 in the clockwise or anti-clockwise direction, causes the movement of the second spacing element 10b along the threaded portion of the connecting rod 14b while the first spacing element 10a remains static in its place.

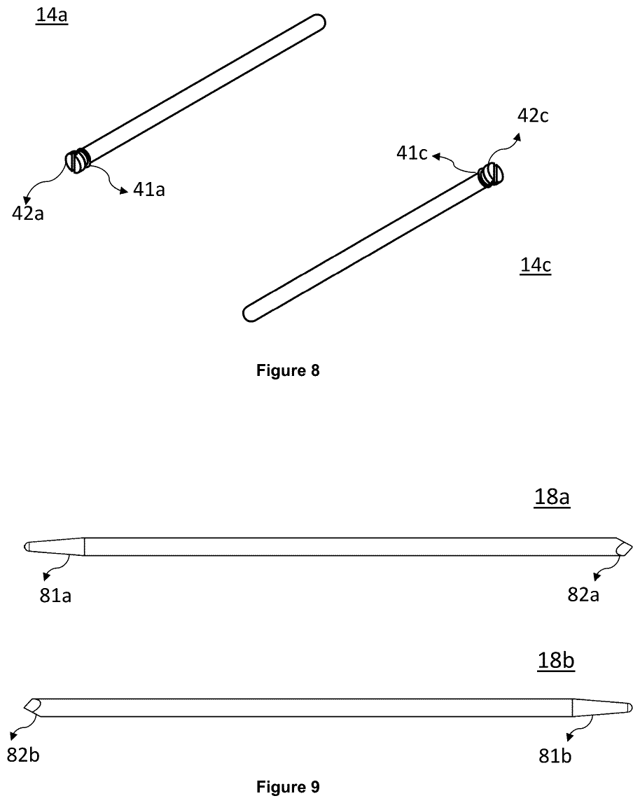

In one embodiment, and as can be better visualized by means of FIGS. 1, 4A, 4B, and 8, the through holes 12a and 12c of the first spacing element 10a, have threaded inner profiles while the through holes 13a and 13c of the second spacing element 10b have smooth inner profiles. In this embodiment, a first support rod 14a is coupled to through hole 12a, of the first spacing element 10a, and to the through hole 13a of the second spacing element 10b, while a second support rod 14c is coupled to through hole 12c, of the first spacing element 10a, and to the through hole 13c of the second spacing element 10b.

In this manner, when the guide device 300 is in its assembled configuration, the first support rod 14a is inserted through the through hole 12a of the first spacing element 10a, and of through hole 13a of the second spacing element 10b until a threaded portion 41a of the first support rod 14a is in contact with the inner threaded profile of the through hole 12a. Accordingly, the second support rod 14c is inserted through the through hole 12c, of the first spacing element 10a, and of through hole 13c of the second spacing element 10b until a threaded portion 41c of the second support rod 14c is in contact with the threaded inner profile of through hole 12c. Each one of the support rods 14a and 14c has an end 42a, 42c adapted to receive torque transmitted by a clamping force of a tool, to provide a firm adjustment between the threaded portions 41a, 41c and the threaded inner profiles.

In this embodiment, the use of support rods 14a, 14c advantageously provides greater robustness and stability to guide device 300 during the use thereof.

As can be seen from FIGS. 1 and 6, the second end 24 of the connecting rod 14b is adapted to receive a bulkhead element, having a diameter at least greater than the diameter of through hole 13b of the second spacing element 10b. In one embodiment, the bulkhead element can have a threaded inner hole, while the second end 24 of the connecting rod 14b can have a threaded hole adapted to receive the bulkhead element. In another embodiment, the bulkhead element can be glued or welded to the second end 24 of the connecting rod 14b.

In these embodiments, the bulkhead element advantageously acts as a barrier to the movement of the second spacing element 10b, preventing the second spacing element 10b from falling off the connecting rod 14b, during the movement of same along the threaded profile of the connecting rod 14b.

According to one embodiment, and as illustrated in FIG. 1, the guide device 300 further comprises a first graded element 11a and a second graded element 11b, each one being respectively coupled on the lower face of the first spacing element 10a and the second spacing element 10b.

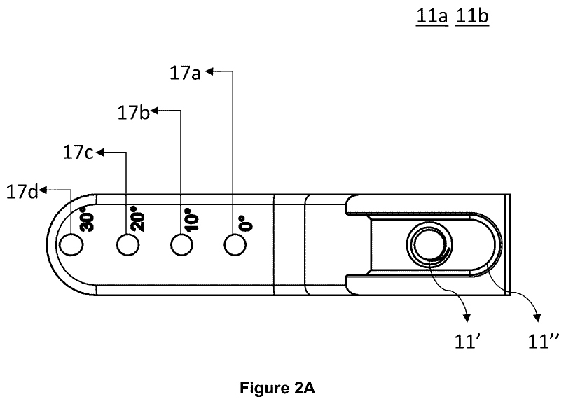

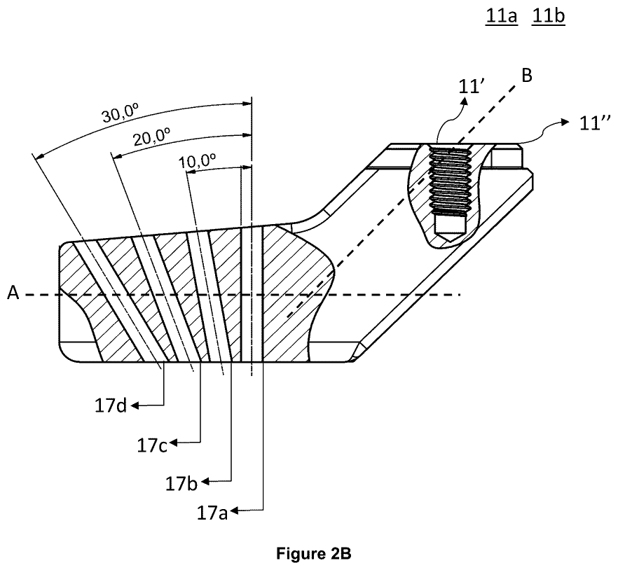

In this embodiment, and as illustrated in FIGS. 2A, 2B, 4A and 4B, the lower face of the first spacing element 10a has a first indentation 10a′ and the back face of the second spacing element 10b has a second indentation 10b′. Additionally, the front face of the first spacing element 10a has a through hole 49a having smooth inner profile, substantially perpendicular to the first indentation 10a′, while the front face of the second spacing element 10b has a through hole 49b having smooth inner profile, substantially perpendicular to the second indentation 10b′.

Each one of the indentations 10a′, 10b′ is adapted to receive, respectively, a first projection 11″ of the first graded element 11a and a second projection 11″ of the second graded element 11b so that the through hole 49a of the first spacing element 10a, is aligned with a first non-through hole 11′ of the first graded element 11′, while the through hole 49b of the second spacing element 10b is aligned with a second non-through hole 11′ of the second graded element 11b.

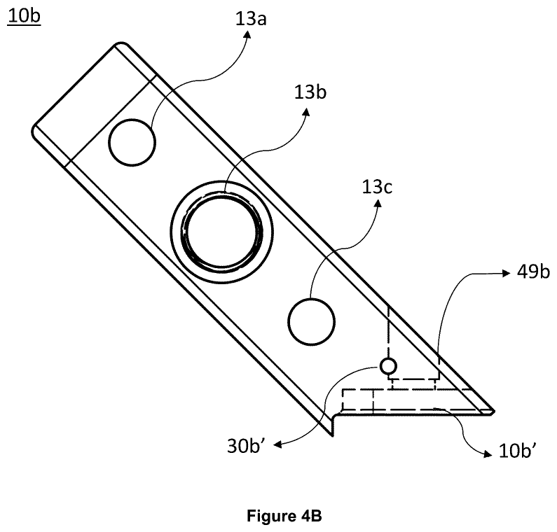

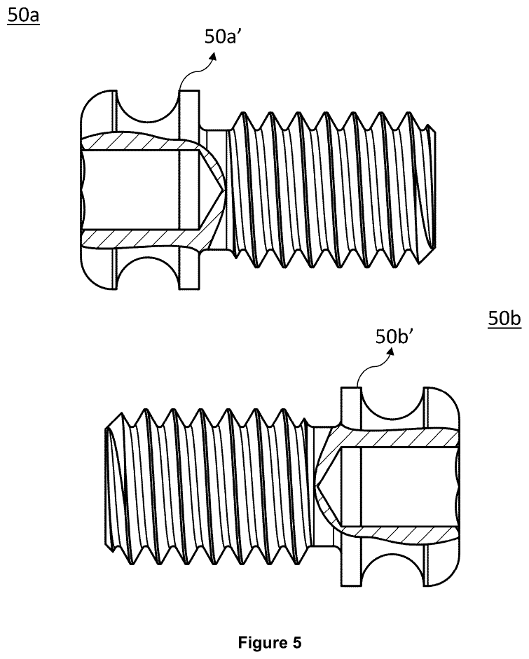

In this embodiment, and as illustrated in FIGS. 2B and 5, the first non-through hole 11′ of the first graded element 11a and the second non-through hole 11′ of the second graded element 11b have a threaded inner profile, each one being adapted to receive, respectfully, a first fastening element 50a and a second fastening element 50b.

In this manner, when the guide device 300 is in its assembled configuration, the first graded element 11a and the second graded element 11b are fixed, respectively, on the lower faces of the first spacing element 10a and the second spacing element 10b, by means of the insertion of the first fastening element 50a, through the through hole 49a of the first spacing element 10a, until a threaded portion of the first fastening element 50a enters into contact with the threaded inner profile of the first non-through hole 11′ of the first graded element 11a and by means of the insertion of the second fastening element 50b through the through hole 49b of the second spacing element 10, until a threaded portion of the second fastening element 50b enters into contact with the threaded inner profile of the second non-through hole 11′ of the second graded element 11b.

In this embodiment, the first fastening element 50a has a grooved profile head 50a′, while the second fastening element 50b′ also has a grooved profile head 50b′. Additionally, as represented in FIGS. 4A and 4B, the first spacing element 10a has a non-through hole 30a′ substantially perpendicular to through hole 49a, while the second spacing element 10b also has a non-through hole 30b′ substantially perpendicular to the through hole 49b.

As illustrated in FIG. 1, when the guide device 300 is in its assembled configuration, the non-through hole 30a′ of the first spacing element 10a is adapted to receive a third lock element 30, while the non-through hole 30b′ of the second spacing element 10b is adapted to receive a fourth lock element (not represented).

In this manner, when inserted in the non-through holes 30a′ and 30b′, part of the third lock element 30 and of the fourth lock element (not represented) rest, respectively, on the grooved profile 50a′ and 50b′ of the first fastening element 50a and of the second fastening element 50b, which advantageously provides greater fastening of the fastening elements 50a and 50b.

In this embodiment, it is apparent that the first spacing element 10a associated to the first graded element 11a and the second spacing element 10b associated to the second graded element 11b are placed parallel to each other.

However, a person skilled in the art will immediately observe that the spacing elements 10a, 10b and the graded elements 11a, 11b, can assume other configurations in other embodiments.

In one embodiment, and as represented in FIGS. 2A and 2B, the first graded element 11a and the second graded element 11b are substantially L-shaped, wherein the first graded element 11a and the second graded element 11b have at least one through hole 17a, 17b, 17c, 17d associated to at least one grading indication engraved close to at least one through hole 17a, 17b, 17c, 17d of each one of the first graded element 11a and the second graded element 11b.

Still in accordance with FIG. 2B, there is illustrated a first reference geometric axis A, and a second reference geometric axis B, which define the substantially L-shape of the first graded element 11a and the second graded element 11b. In this embodiment, an opening angle of the first reference geometric axis A, relative to the second reference geometric axis B, is obtuse, the angular value being preferably comprised in the range of 90° to 120°.

In this embodiment, the at least one through hole 17a, 17b, 17c, 17d of each one of the first graded element 11a and the second graded element 11b, varies progressively in angulation relative to the grading. For example, the at least one through hole 17a, 17b, 17c, 17d is a first 17a, second 17b, third 17c and fourth 17d through hole. Accordingly, the grading indication engraved close to each one of the through holes 17a, 17b, 17c, 17d and progressive variation in angulation are related to the first through hole 17a in 0°, to the second through hole 17b in 10°, to the third through hole 17c in 20° and to the fourth through hole 17d in 30°.

In another embodiment, the grading indication engraved close to each one of the through holes 17a, 17b, 17c, 17d and the progressive variation can be of 0° to 30°, so that there exist 31 through holes, each one varying progressively in angulation in 1 degree of difference relative to the previous through hole, which gives the surgeon greater possibility in choice of the angulation adjustment during the bone correction procedure. However, it will be understood by a person skilled in the art that the first graded element 11a and the second graded element 11b can contain any number of through holes.

Additionally, due to the previously described manner of fastening, respectively, the first graded element 11a and the second graded element 11b in the first spacing element 10a and in the second spacing element 10b, the present invention advantageously provides a manner to unpin said graded elements 11a and 11b, in case it is necessary to exchange them for a variation containing a larger or smaller amount of through holes.

In another embodiment, the first graded element 11a and the first spacing element 10a, as well as the second graded element 11b and the second spacing element 10b, can be integrally formed in monoblock parts.

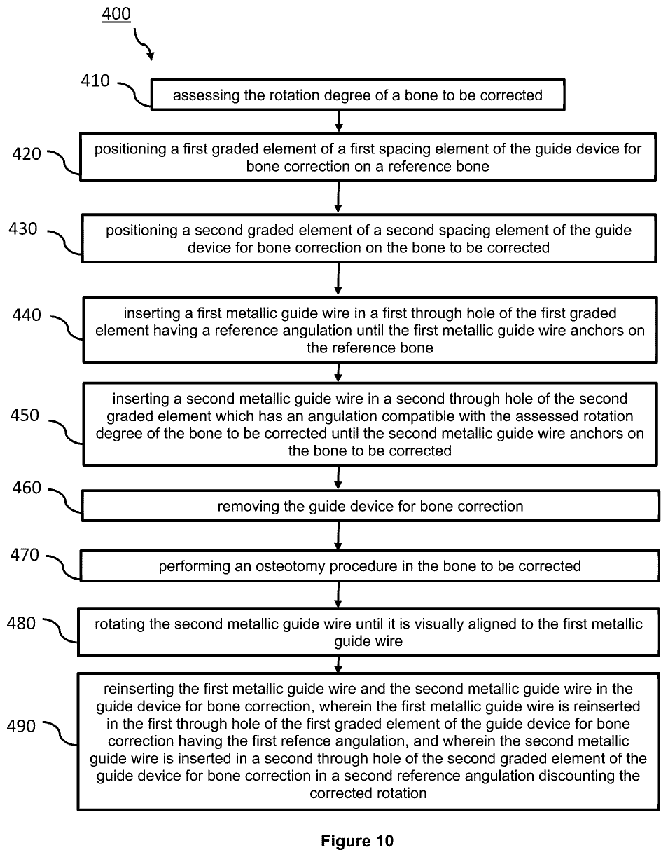

As illustrated in FIG. 9, the first 17a, second 17b, third 17c and fourth 17d through hole of each one of the first graded element 11a and the second graded element 11b are adapted to receive, respectively, a first metallic guide wire 18a and a second metallic guide wire 18b. In this embodiment, the first metallic guide wire 18a and the second metallic guide wire 18b have, respectively, a first substantially frustoconical end 81a, 81b adapted to be driven by an orthopedic drill, and a second piercing end 82a, 82b.

In one embodiment, the first graded element 11a and the second graded element 11b are manufactured in polymeric material, preferably in polyacetal, while the remaining components of the guide device 300 are made in one of hardened stainless steel 420B, stainless steel 420B— ASTM F899, stainless steel— ASTM F138, whereby they can be thermally treated according to ETS-28M, with a hardness between 49 and 52 HRC.

In this embodiment, the metallic guide wires 18a, 18b can be made in stainless steel— ASTM F138 given its biocompatibility and possibility of sterilization. The remaining components of the guide device 300, except the first graded element 11a and the second graded element 11b, can be made in hardened 420B stainless steel or 420B— ASTM F899 stainless steel, due to their resistance to wear and corrosion, apart from the possibility of sterilization after use.

In another embodiment, all the components of the guide device 300 are made from a single material.

In another embodiment, and as illustrated in FIG. 10, the bone correction method 400 disclosed by the present invention refers to a rotational correction of the head of a first metatarsal bone plantar flexed, during a distal percutaneous osteotomy of the first metatarsal bone with rotation of the metatarsal head, and preferably comprises the use of the guide device 300 for the bone correction procedure, previously cited.

In this embodiment, the bone correction method 400 comprises assessing 410 the degree of plantar flexion of the head of the first metatarsal bone, in a preoperative step by means of obtaining images such as X-rays or tomography with load, so that the surgeon starts an intraoperative step, aware of how many degrees the head of the first metatarsal bone must be rotated to obtain a precise alignment.

Next, during the intraoperative step the method 400 comprises positioning 420 the lower face of the first graded element 11a of the first spacing element 10a of the guide device 300 on a reference bone, preferably in the medial cuneiform bone or in another anatomical safety zone that is, an area of the body that can receive a metallic guide wire 18a, without interfering in nerves, arteries, etc. of the patient).

The method 400 further comprises, positioning 430 the lower face of the second graded element 11b of the second spacing element 10b of the guide device 300 on the head of the first plantar flexed metatarsal bone.

In this sense, the substantially L-shape of the first graded element 11a and of the second graded element 11b advantageously allows the lower face of each one of said graded elements 11a and 11b to rest in a stable manner on the cuneiform medial bone and on the head of the first metatarsal bone plantar flexed.

The method 400 further comprises inserting 440 the second piercing end 82a of the first metallic guide wire 18a in one of the through holes 17a, 17b, 17c, 17d of the first graded element 11a which has a first reference angulation until the first metallic guide wire 18a anchors in the medial cuneiform bone.

In this sense, the reference angle is preferably 0°. In this manner, the second piercing end 82a of the first metallic guide 18a is preferably inserted through the first through hole 17a which has an angulation in 0°, wherein the anchoring occurs by means of the action of the first substantially frustoconical end 81a, by orthopedic drill.

The method 400 further comprises inserting 450 the second piercing end 82b of the second metallic guide wire 18b in one of the through holes 17a, 17b, 17c, 17d of the second graded element 11b which has an angulation compatible with the degree of plantar flexion of the head of the first metatarsal bone assessed, until the second metallic guide wire 18b anchors on the head of the first metatarsal bone.

In an analogous manner, the anchoring is performed by means of the action of the first substantially frustoconical end 81b, by the orthopedic drill.

The method 400 further comprises, removing 460 the guide device 300 for bone correction, sliding same through the extension of the first metallic guide wire 18a and the second metallic guide wire 18b until it escapes through the substantially frustoconical ends 81a, 81b. In this sense, it must be noted that the frustoconical geometric profile advantageously allows the guide device 300 to be removed with greater ease and less interference between the metallic guide wires 18a, 18b and the through holes 17a, 17b, 17c, 17d.

The method 400 further comprises performing 470 an osteotomy procedure in the first metatarsal bone, originating a bone fragment that is anchored to the second metallic guide wire 18b. Preferably, the procedure is an extracapsular transverse percutaneous osteotomy, performed by means of an access at the base of the exostosis. The osteotomy is carried out by means of a drill or suitable equipment until there occurs the complete separation of the head of the first metatarsal bone from the rest of the first metatarsal bone.

Next, the method 400 comprises the positioning of the remaining guide wires wherein a proximal guide wire relative to the base of the first metatarsal bone is inserted through the medial and lateral cortical bone, while a distal guide wire relative to the base of the first metatarsal bone is inserted parallel to the proximal guide wire, both by means of an orthopedic drill.

The method 400 further comprises rotating 480 the second metallic guide wire 18b until it is visually aligned to the first metallic guide wire 18a and reinserting 490 the first metallic guide wire 18a and the second metallic guide wire 18b in the guide device 300, wherein the first metallic guide wire 18a receives the first through hole 17a of the first graded element 11a of the guide device 300 in reference angulation 0° and wherein the second metallic guide wire 18b receives the second through hole 17a of the second graded element 11b of the guide device 300 in a second reference angulation, discounting the corrected rotation, and therefore, correcting the rotation of the bone to be corrected. Preferably, the second reference angulation is equal to the first reference angulation.

The method 400 further comprises the insertion of an intramedullary equipment in the first metatarsal bone while laterally sliding the head of the first metatarsal bone. After the correction of the plantar flexion of the head of the first metatarsal bone and the valgus parameter, the guide wires are advanced by means of the action of the orthopedic drill, the intramedullary equipment is removed, and bone fixation screws are inserted through the guide wires, to perform the osteosynthesis. The guide device 300 and the metallic guide wires 18a, 18b are subsequently removed.

The method 400 continues with the medial metaphyseal nozzle resection, followed by an Akin osteotomy, the fixation of the bone sections of the proximal phalanx I by means of bone screws, and finally the exostectomy of the head of the first metatarsal bone.

Although the description of the particular embodiments above refers to certain embodiments, the present invention can present modifications in its manner of implementation, so the scope of protection of the invention is limited only by the contents of the attached claims, including therein the possible equivalent variations.

1. A guide device (300) for bone correction procedure, comprising:
a first spacing element (10a) and a second spacing element (10b), each one of the first spacing element (10a) and the second spacing element (10b) having at least one through hole (12a, 13a, 12b, 13b, 12c, 13c);
at least one connecting rod (1b) coupled to the at least one through hole (12a, 13a, 12b, 13b, 12c, 13c) of the first spacing element (10a) and the at least one through hole (12a, 13a, 12b, 13b, 12c, 13c) of the second spacing element (10b), and
a first graded element (11a) and a second graded element (11b), being each one respectively coupled to the first spacing element (10a) and to the second spacing element (10b), each one of the first graded element (11a) and the second graded element (11b) having at least one through hole (17a, 17b, 17c, 17d) associated to at least one grading.

2. The device (300) according to claim 1, wherein the at least one through hole (12b) of the first spacing element (10a) has a smooth inner profile in contact with a smooth portion of the at least one connecting rod (14b).

3. The device (300) according to claim 1, wherein the at least one through hole (13b) of the second spacing element (10b) has a threaded inner profile in contact with a threaded portion of the at least one connecting rod (14b).

4. The device (300) according to claim 1, wherein the at least one connecting rod (14b) comprises a handle (15).

5. The device (300) according to claim 4, wherein the first spacing element (10a) is arranged at a proximal end relative to the handle (15).

6. The device (300) according to claim 5, wherein the first spacing element (10a) is fixed on the proximal end by means of at least one lock element (40).

7. The device (300) according to claim 4, wherein the second spacing element (10b) is arranged at a distal end relative to the handle (15).

8. The device (300) according to claim 4, wherein the second spacing element (10b) is movable along the at least one connecting rod (14b) by action of the handle (15).

9. The device (300) according to claim 1, further comprising at least one support rod (14a, 14c) coupled to one of the at least one through hole (12a, 13a) of the first spacing element (10a) and one of the at least one through hole (12c, 13c) of the second spacing element (10b).

10. The device (300) according to claim 1, wherein the at least one through hole (17a, 17b, 17c, 17d) of each one of the first graded element (11a) and the second graded element (11b) have a progressive variation in angulation with respect to grading.

11. The device (300) according to claim 10, wherein the at least one through hole (17a, 17b, 17c, 17d) comprises a first (17a), second (17b), third (17c) and fourth (17d) through hole, and the progressive variation in angulation related to the first through hole (17a) is 0°, the progressive variation in angulation related to the second through hole (17b) is 10°, the progressive variation in angulation related to the third through hole (17c) is 20°, and the progressive variation in angulation related to the fourth through hole (17d) is 30°.

12. The device (300) according to claim 10, wherein the progressive variation in angulation is 0° to 30°.

13. The device (300) according to claim 10, wherein the at least one through hole (17a, 17b, 17c, 17d) of each one of the first graded element (11a) and the second graded element (11b) is adapted to receive, respectively, a first metallic guide wire (18a) and a second metallic guide wire (18b).

14. A bone correction method (400) comprising the steps of:
assessing (410) a rotation degree of a bone to be corrected;
positioning (420) a first graded element (11a) of a first spacing element (10a) of a guide device (300) for bone correction on a reference bone;
positioning (430) a second graded element (11b) of a second spacing element (10b) of the guide device (300) for bone correction on the bone to be corrected;
inserting (440) a first metallic guide wire (18a) in a first through hole (17a, 17b, 17c, 17d) of the first graded element (11a) which has a first reference angulation until the first metallic guide wire (18a) anchors on the reference bone;
inserting (450) a second metallic guide wire (18b) in a second through hole (17a, 17b, 17c, 17d) of the second graded element (11b) that has an angulation compatible with the assessed rotation degree of the bone to be corrected until the second metallic guide wire (18b) anchors on the bone to be corrected;
removing (460) the guide device (300) for bone correction;
performing (470) an osteotomy procedure on the bone to be corrected;
rotating (480) the second metallic guide wire (18b) until it is visually aligned to the first metallic guide wire (18a); and
reinserting (490) the first metallic guide wire (18a) and the second metallic guide wire (18b) in the guide device (300) for bone correction, wherein the first metallic guide wire (18a) is reinserted in the first through hole (17a, 17b, 17c, 17d) of the first graded element (11a) of the guide device (300) for bone correction which has a reference angulation, and wherein the second metallic guide wire (18b) is inserted in a second through hole (17a, 17b, 17c, 17d) of the second graded element (11b) of the guide device (300) for bone correction in a second reference angulation, discounting the corrected rotation.
